(19) United States
(12) Patent Application Publication
(10) Pub. No.: US 2012/0310178 A1
(43) Pub. Date: Dec. 6, 2012

(54) VALVE ASSEMBLY

(75) Inventors: Louis B. Goode, Cranberry Township, PA (US); Robert Booker, Vandergrift, PA (US)

(73) Assignee: Cook Medical Technologies LLC, Bloomington, IN (US)

(21) Appl. No.: 13/150,621

(22) Filed: Jun. 1, 2011

(51) Int. Cl. A61M 5/00

(52) U.S. Cl. 604/246

(57) ABSTRACT

A valve assembly for controlling a flow of fluid includes a frame member and dual tubular valves arranged along the length of the frame member. The frame member includes fixed proximal and distal portions, and a rotatable intermediate portion. One valve is affixed between the proximal and intermediate portions, and the other valve is affixed between the intermediate and distal portions. The intermediate portion is selectively rotatable such that when one of the valves is in an open condition, the other valve is in a constricted condition.

### BACKGROUND

1. Technical Field

The present invention relates to a valve assembly. More particularly, the invention relates to a valve assembly incorporating dual valves for controlling the flow of fluid through a medical device.

2. Background Information

A variety of well-known medical procedures are initiated by introducing an interventional device such as a catheter, sheath, trocar, and the like into a vessel in a patient's body. Typical procedures for introducing an interventional device into a blood vessel include the well-known Seldinger technique. In the Seldinger technique, a needle is injected into a blood vessel, and a wire guide is inserted into the vessel through a bore of the needle. The needle is withdrawn, and a dilator/introducer sheath assembly is inserted over the wire guide. The introducer sheath typically includes a hemostatic valve, through which the dilator passes. Following proper placement of the introducer sheath, the dilator is removed. The interventional device may then be inserted through the sheath and hemostatic valve into the vessel.

As the interventional device is introduced into the vessel, care must be taken to avoid the undesirable leakage of blood or other bodily fluids, or a cavity-pressurizing gas, from the patient. Similarly, care must be taken to avoid the undesirable introduction or leakage of air into the vessel. As procedures for introducing catheters and other interventional devices have become more widely accepted, the procedures associated with their use have become more diverse, and the variety of sizes and types of such introducer devices has grown dramatically. As a result, the risk of inward or outward leakage of fluids has increased, along with the necessity to maintain vigilance to minimize the possibility of such leakage.

One known way to minimize leakage is to provide one or more disk-like gaskets in an elongated passageway of a device through which fluids may be controllably passed into or out of the body. Such disks have opposing surfaces and often include one or more slits that extend partially across each of the surfaces and inwardly toward the interior of the disk. A generally axial opening is provided between the slits to provide a sealable path for insertion of an interventional device through the disks. Examples of such disks are described, e.g., in U.S. Pat. Nos. 5,006,113 and 6,416,499, incorporated by reference herein. These disks are generally effective for sealing large diameter devices, but may be less effective for sealing smaller diameter devices. This may be especially true when a smaller diameter device is introduced through a disk following the earlier passage of a larger diameter device. In this instance, the valve may not re-seal in a desirable manner during passage of the smaller diameter device. In addition, such disks may exert an undesirable amount of force on delicate devices passing therethrough, thereby increasing a risk of damage to such devices.

Another type of valve that may be used for sealing elongated passages in a medical device to prevent passage of fluids is commonly referred to as an iris valve. Iris valves are described, e.g., in U.S. Pat. Nos. 5,158,553 and 7,172,580, incorporated by reference herein. An iris valve may comprise a valve hub that is joined to a catheter-type device, and a knob that is rotatably engaged with the hub. An elastomeric sleeve having an elongated passageway therethrough is positioned in an opening through the interior of the valve body. The opposing axial ends of the elastomeric sleeve are joined to the hub and the rotatable knob, respectively. When the rotatable knob is rotated in a first direction, the passageway of the elastomeric sleeve is fully opened. When the knob is rotated in a second direction opposite the first direction, the elastomeric sleeve is twisted intermediate the two ends to effect closure of all or part of the elongated passageway.

Although suitably-sized iris valves may be utilized with sheaths of virtually all diameters and compositions, such valves are particularly effective when larger diameter sheaths (e.g., sheaths having French sizes of from about 5 Fr. To about 18 Fr.) are introduced into larger body vessels having high blood flow, such as the jugular vein. In such large vessels, blood loss can become a major issue when a medical interventional device, such as a dilator, is introduced through the lumen of the sheath. Thus, a suitable valve, such as an iris valve, may be provided interiorly of the sheath to reduce such loss. An iris valve may be opened to permit passage of the device therethrough, and closed around the device to prevent leakage of blood through the sheath. The iris valve can be controllably closed around the device, regardless of the diameter of the device. The amount of tension exerted on the interventional device can be controlled such that the interventional device can generally be inserted, maneuvered, and removed without major blood loss. In addition, the ability to control the amount of tension exerted on the interventional device allows the introduction of smaller diameter or otherwise delicate devices through the valve at a reduced risk of damage.

Notwithstanding the advantages realized with the use of an iris valve, such valves must nonetheless be opened and closed to permit passage of the interventional device. When the device is opened to permit passage of the device, blood may escape through the opened valve. Although the amount of blood loss may not be significant, any such loss is disadvantageous, not only to the patient, but also to the health care worked exposed to the leaked blood.

It would be desirable to provide a valve assembly that overcomes the problems associated with prior art valves, and that is effective for minimizing the amount of blood loss through the sheath.

### BRIEF SUMMARY

The shortcomings of the prior art are addressed by the present valve assembly. In one form, a valve assembly for controlling a flow of fluid is provided. The valve assembly comprises a valve frame comprising a proximal portion, an intermediate portion, and a distal portion. The proximal portion and the distal portion have a substantially fixed orientation along a length of the valve frame, and the intermediate portion is selectively rotatable between a first position and a second position. A first valve member has a proximal end, a distal end, and a passageway therebetween. The proximal end is secured to the valve frame proximal portion, and the distal end is secured to the intermediate portion. The first valve member is arranged relative to the valve frame proximal portion and intermediate portion such that the passageway is in an open condition when the intermediate portion is in the first position, and the passageway is in a constricted condition when the intermediate portion is in the second position. A second valve member has a proximal end, a distal end, and a passageway therebetween. The proximal end is secured to the intermediate portion, and the distal end is secured to the valve frame distal portion. The second valve member is arranged relative to the valve frame distal portion and intermediate portion such that the passageway is in a constricted condition when the intermediate portion is in the first position, and the passageway is in an open condition when the intermediate portion is in the second position.

In another form, a dual valve assembly controls a flow of fluid. A housing comprises a proximal hub member, an intermediate hub member, and a distal hub member. The proximal hub member, intermediate hub member, and distal hub member are configured and aligned in an interior space of the housing for passage of an interventional device. The proximal hub member and distal hub member have a substantially fixed orientation in the housing interior space, and the intermediate hub member is rotatable between a first position and a second position in the housing interior space. A first valve sleeve has a proximal end, a distal end, and a passageway therebetween for passage of the interventional device. The proximal end is secured to the proximal hub member, and the distal end is secured to the intermediate hub member. The first valve sleeve is arranged relative to the proximal hub member and intermediate hub member such that the passageway is in an open condition for passage of the interventional device when the intermediate hub member is in the first position, and the passageway sealingly constricts to a closed condition when the intermediate hub member is in the second position. A second valve sleeve has a proximal end, a distal end, and a passageway therebetween for passage of the interventional device. The proximal end is secured to the intermediate hub member, and the distal end is secured to the valve frame distal hub member. The second valve sleeve is arranged relative to the valve frame distal hub member and the intermediate hub member such that the passageway sealingly constricts to a closed condition when the intermediate hub member is in the first position, and the passageway is in an open condition for passage of the interventional device when the intermediate hub member is in the second position.

### BRIEF DESCRIPTION OF THE DRAWINGS

FIG. 1 is a side view of a valve assembly, according to an embodiment of the invention;

FIG. 2A is a side view of the valve assembly of FIG. 1, including a housing;

FIG. 2B is a side view of the valve assembly rotated 90 degrees from the orientation of FIG. 2A;

FIG. 2C is a side view of the valve assembly rotated 180 degrees from the orientation of FIG. 2A;

FIG. 3 is a side view of the valve assembly of FIG. 1, in combination with a dilator, a sheath and a flush assembly;

FIG. 4 is an exploded view of the valve assembly of FIG. 1, also illustrating the sheath and flush assembly; and

FIG. 5 is a side view of the valve assembly of FIG. 2A following rotation of the valve control knob.

### DESCRIPTION OF THE PREFERRED EMBODIMENTS

For purposes of promoting an understanding of the present invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the valve assembly, as well as the axial ends of various component features. The term “proximal” is used in its conventional sense to refer to the end of the valve assembly (or component thereof) that is closest to the operator during use. The term “distal” is used in its conventional sense to refer to the end of the valve assembly (or component thereof) that is initially inserted into the patient, or that is closest to the patient during use.

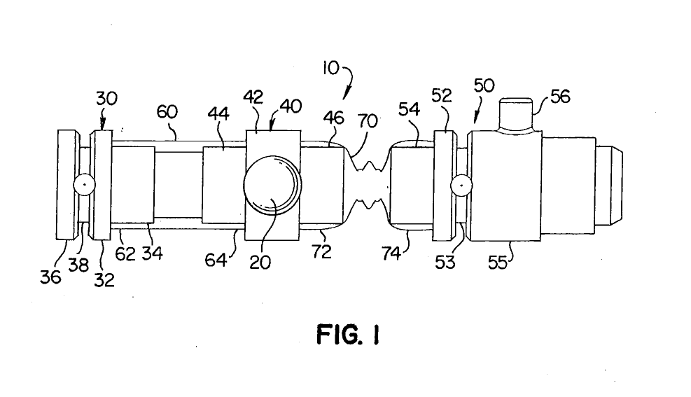

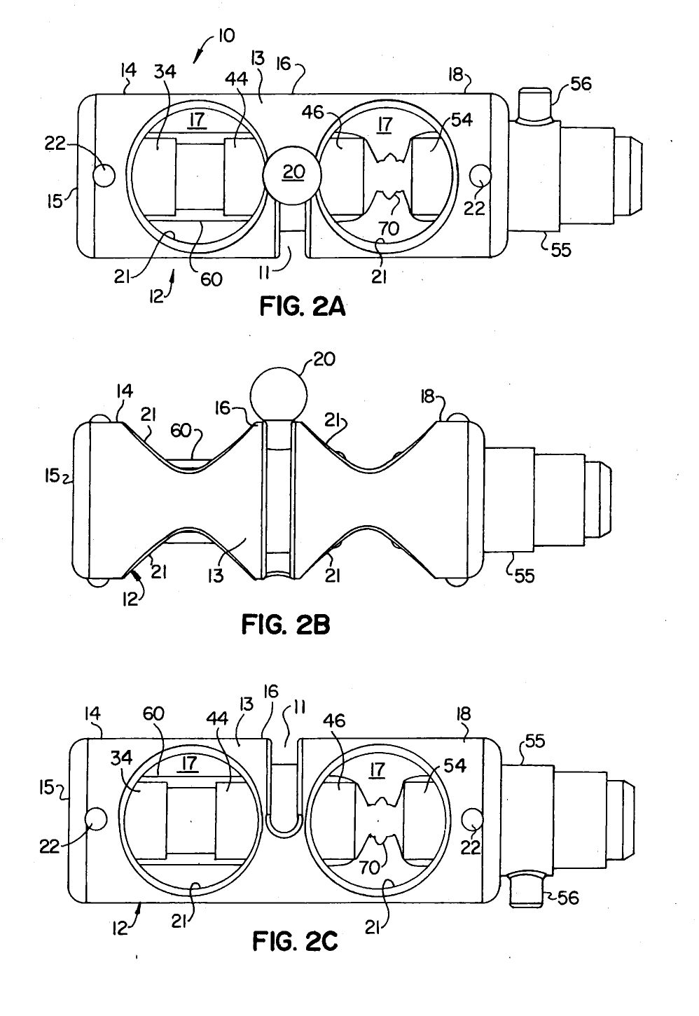

FIGS. 1 and 2A-2C illustrate side views of a valve assembly 10 according to an embodiment of the invention. Valve assembly 10 includes a frame member 12 comprising a proximal hub member 30, an intermediate hub member 40, a distal hub member 50, and optionally, a housing 13 (FIGS. 2A-2C) for retaining the hub members 30, 40, 50. In the embodiment shown, valve assembly 10 comprises a first, or proximal, valve member 60, and a second, or distal, valve member 70. In this embodiment, valve members 60, 70 comprise elastomeric, generally tubular sleeves. In FIG. 1 first valve member 60 is shown in an open condition, and second valve member 70 is shown in a constricted, or closed, condition. Further discussion of the structure and operation of the valve members will be provided herein.

FIG. 1 is a side view of valve assembly 10. Housing 13 has been omitted from FIG. 1 to better illustrate other features of the valve assembly. In the non-limiting embodiment shown, proximal hub member 30 comprises a larger diameter portion 32, and a smaller diameter generally cylindrical portion 34 extending in the distal direction from larger diameter portion 32. Proximal end 62 of first valve member 60 fits over proximal hub smaller diameter portion 34. In the embodiment shown, hub member 30 also includes larger diameter portion 36 and smaller diameter portion, or slot, 38.

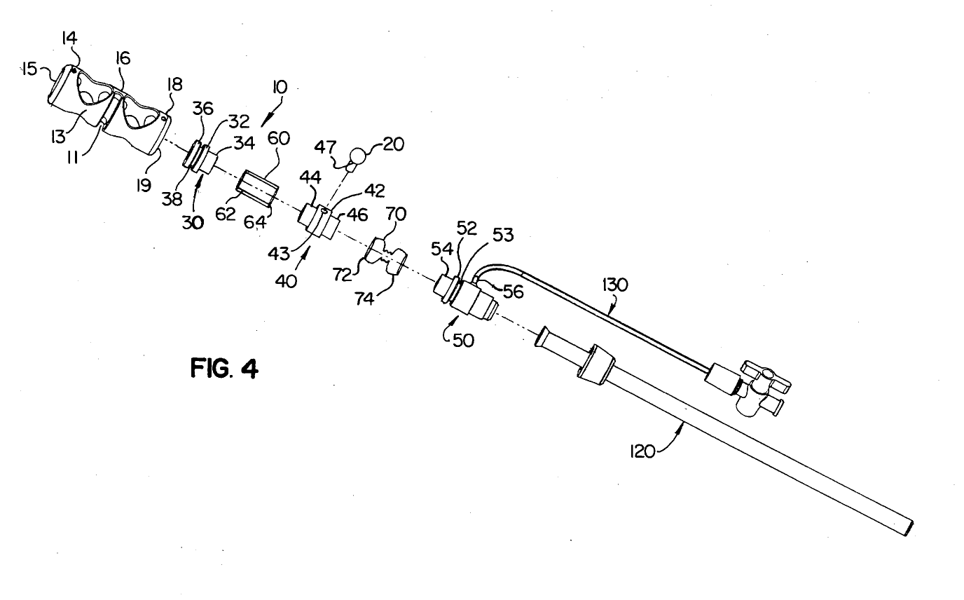

Intermediate hub member 40 includes a larger diameter middle portion 42, and respective smaller diameter proximal 44 and distal 46 portions extending axially from respective sides of larger diameter middle portion 42. Distal end 64 of first valve member 60 fits over smaller diameter proximal portion 44. Proximal end 72 of second valve member 70 fits over smaller diameter distal portion 46. Larger diameter portion 42 includes an aperture 43 (FIG. 4) sized to receive an end 47 of a valve control member, such as knob 20.

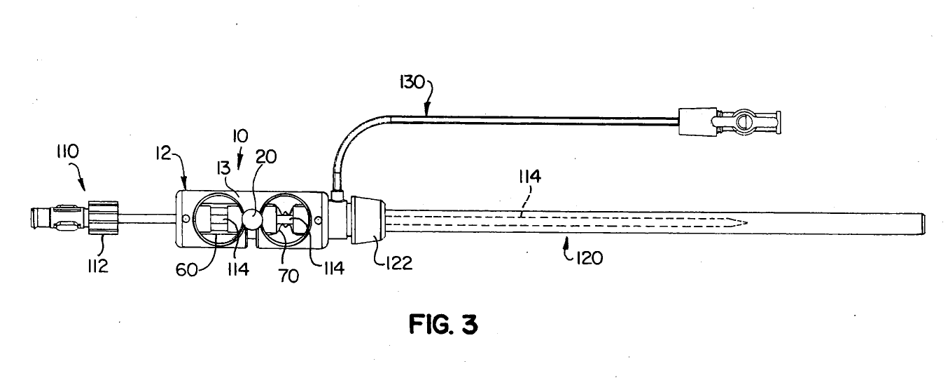

Distal hub member 50 comprises a larger diameter portion 52, and a smaller diameter generally cylindrical portion 54 extending in the proximal direction from larger diameter portion 52. Distal end 74 of second valve member 70 fits over distal hub smaller diameter portion 54. The respective ends of valve member 60, 70 are secured to the respective hub member portion in a suitable manner to inhibit disengagement therefrom, e.g., by a friction fit and/or use of a biologically suitable adhesive. In the embodiment shown, hub member 50 also includes a larger diameter flush port hub member 55 and a smaller diameter portion, or slot, 53. Flush port hub member 55 includes a port 56 configured to receive, e.g., a conventional flush assembly 130 (FIGS. 3, 4) or other apparatus commonly used in conjunction with a valve member.

FIGS. 2A-2C illustrate side views of the dual valve assembly 10. FIG. 2A is a side view of one side of dual valve assembly 10. FIG. 2B is a side view of the entire dual valve assembly rotated 90 degrees from the orientation of FIG. 2A. FIG. 2C is a side view of the dual valve assembly rotated 180 degrees from the orientation of FIG. 2A. In the embodiment shown, housing 13 includes a proximal portion 14, an intermediate portion 16, and a distal portion 18. In the preferred embodiment shown, housing 13 has a generally cylindrical configuration, and includes a proximal opening 15, a distal opening 19 (FIG. 4), and an interior space 17. A pair of windows 21 may be positioned along the length of each side of housing 13 to provide a view of respective valve members 60, 70. Housing 13 is also provided with a slot 11 extending along approximately one-half of the circumference of intermediate portion 16. Control knob 20 rides along slot 11, in a manner to be described.

Housing 13 is configured to securely receive and retain proximal hub member 30, intermediate hub member 40, and distal hub member 50. Those skilled in the art can readily fashion suitable means for securing hub members 30, 40, 50 in housing 13. In the embodiment shown, hub members 30, 50 are secured by screws 22. Preferably, screws 22 are provided along each axial side of housing 13 to engage housing proximal portion 14 with small diameter portion 38 of hub 30, and distal portion 18 with small diameter portion 53 of hub 50, respectively (FIGS. 1, 4). Although in the illustrated embodiments frame member 12 comprises housing 13 for retaining hub members 30, 40, 50 in the orientation described, those skilled in the art will appreciate that other suitable structure for retaining the hub members in this orientation may be substituted for housing 13.

In FIG. 3, dual valve assembly 10 is shown in combination with optional features comprising a dilator 110, a sheath 120, and a flush assembly 130. Dilator 110 has a hub 112, and an elongated tubular portion 114 extending distally from hub 112. Dilator tubular portion 114, extending interiorly of sheath 120, is shown in phantom in FIG. 3. Nose portion 122 is provided to releasably engage sheath 120 with distal hub member 50. For ease in viewing the respective components, Fig, 4 is an exploded view illustrating the dual valve assembly 10 of FIG. 1, in combination with sheath 120 and flush assembly 130.

Valve members 60, 70 may be of a type commonly referred to in the medical arts as iris valves. Valves of this general type are described above, and e.g., in the incorporated-by-reference U.S. Pat. Nos. 5,158,553 and 7,172,580. In the embodiments shown, each of valve members 60, 70 comprises a generally cylindrical elastomeric sleeve. However, this configuration is not crucial, and the valve members may alternatively have other configurations, such as the flanges, and/or the accordion-like and hourglass-like shapes shown in FIGS. 16 and 17 of the incorporated-by-reference '580 patent. As a still further alternative, the valve members can be provided with annular or ribbed sealing structures, as further described in the '580 patent. In one embodiment, valves 60, 70 can have respective inner diameters from about 0.25 to about 0.50 inch, and can have respective lengths from about 0.50 to about 1.0 inch.

Valve members 60, 70 are preferably elastomeric, and more preferably, may be formed of injection molded silicone. A non-limiting list of other suitable materials for use in forming the valve member includes isoprene, latex and various rubber or polymeric compositions. For purposes of the present invention, the durometer of the valve member should be considerably less than the durometer of the housing and hubs, resulting in a valve sleeve that is softer and more flexible than the remaining structure. If desired, the valve member, or preferably, the internal surface only of the valve member, can be coated with a lubricious coating, such as parylene, to improve the lubricity of the surface and for ease of movement of the interventional device once the device is received in the valve.

Housing 13 and hub members 30, 40, and 50 are preferably formed of conventional polymeric materials well known for such use in the medical arts, such as acetals, polycarbonates, and acrylonitrile butadiene styrene (ABS). As illustrated, each of the aforementioned constituents includes a hollowed-out center portion to enable passage of an interventional device therethrough. The hub members may be formed from conventional techniques, such as injection molding or machining.

The following discussion describes one manner of using dual valve assembly 10. Iris-type valves are generally effective for sealing sheaths of virtually all sizes and compositions. Such valves are particularly effective for sealing larger diameter sheaths. However, there are certain limitations when using iris valves with such sheaths. For example, when passing an interventional device through a valve in the open position, bodily fluids may leak through the valve as the interventional device is advanced along the open valve. Once the interventional device has been properly positioned, the valve can be closed or constricted around the device to prevent further leakage. However, an undesirable amount of fluid may have leaked through the valve prior to closure. Additional fluid may leak as the valve is opened to allow removal of the device. In order to avoid this leakage, attempts may be made to force the interventional device through a closed valve. However, in this instance, either the device, or the valve, is subject to damage due to the insertion force exerted by the device against the constricted flexible valve member. This can result in a decreased lifetime of the valve, and in some instances, outright valve failure. In addition, when the interventional device has a small diameter or is otherwise of delicate construction, the interventional device is at increased risk of damage during passage through a closed valve.

Use of a multi-valve assembly, such as the dual valve assembly described and illustrated herein, enables the artisan to overcome these limitations. As illustrated in the example referenced herein, dilator 110 is advanced through the dual valve assembly 10 such that leading end 114 extends into sheath 120, as shown in FIG. 3. Initially, in order to insert the dilator the intermediate hub member 40 is rotated, e.g., via the rotatable valve control member such as knob 20, to the position shown in FIGS. 1-3. In this instance, proximal valve member 60 is in the open condition, and distal valve member 70 is in the closed, or constricted, condition as shown. At this time, the leading (distal) end of dilator tubular member 114 is freely passable through open valve member 60. The closed condition of valve member 70 prevents leakage of bodily fluids back through open valve member 60. This closed valve also restricts, or at least inhibits, passage of tubular member 114 through valve member 70.

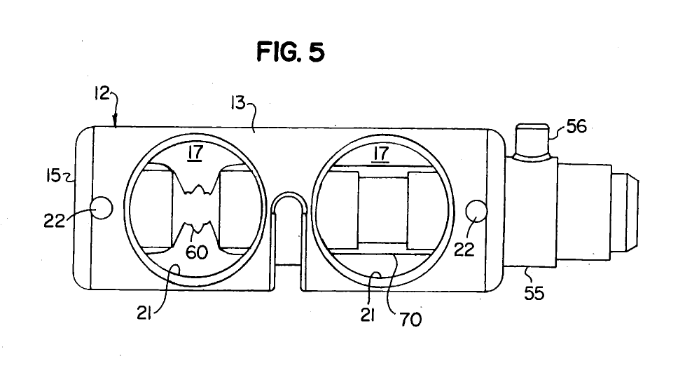

In order to facilitate leak-free insertion of the leading end of the dilator tubular member 114 through housing 13, intermediate hub 40 is rotated from the first position, e.g., as shown in FIGS. 1-3, to a second position, e.g., as shown in FIG. 5. In the embodiment shown, this rotation is effected by riding the knob 20 along housing slot 11. Rotation of knob 20 results in the rotation of intermediate hub member 40, and thereby, intermediate hub member middle portion 42, proximal portion 44, and distal portion 46. Upon such rotation to the orientation of FIG. 5, valve member 60 is twisted into the closed, or constricted, condition, and valve member 70 is now in the open condition as shown.

The degree of closure of proximal valve member 60 around dilator 114, and therefore the amount of tension exerted by the constricted valve member 60 on the dilator during insertion, may be controlled by selectively adjusting the degree of rotation of control knob 20. As a result, one valve member need not be fully opened, and the other valve member need not be fully closed, or constricted, at any one time. Rather, adjusting the degree of rotation of knob 20 enables the user to vary the amount of closure such that, e.g., proximal valve member 60 may be substantially closed or constricted, to inhibit leakage, and yet still permit passage of dilator 114 therethrough. At this time, distal valve member 70 may be substantially (but less than 100%) opened. The ability to simultaneously control the degree of closure of valve members 60, 70 allows dilator 114 to be passed through the respective valve members, and at the same time, minimizes leakage of blood therethrough.

Although the valve assembly has been described herein as a dual valve assembly, those skilled in the art will appreciate that other numbers of valves may be substituted. Thus, e.g., three, four, or more valves may be aligned as described herein upon minimal re-design of the assembly described herein to account for the additional valves.

While these features have been disclosed in connection with the illustrated preferred embodiments, other embodiments of the invention will be apparent to those skilled in the art that come within the spirit of the invention as defined in the following claims.

1. A valve assembly for controlling a flow of fluid, the assembly comprising:
a valve frame comprising a proximal portion, an intermediate portion, and a distal portion, said proximal portion and said distal portion having a substantially fixed orientation along a length of said valve frame, said intermediate portion selectively rotatable between a first position and a second position;
a first valve member having a proximal end, a distal end, and a passageway therebetween, said proximal end secured to said valve frame proximal portion, and said distal end secured to said intermediate portion, said first valve member arranged relative to said valve frame proximal portion and intermediate portion such that said passageway is in an open condition when said intermediate portion is in said first position, and said passageway is in a constricted condition when said intermediate portion is in said second position; and
a second valve member having a proximal end, a distal end, and a passageway therebetween, said proximal end secured to said intermediate portion, and said distal end secured to said valve frame distal portion, said second valve member arranged relative to said valve frame distal portion and intermediate portion such that said passageway is in a constricted condition when said intermediate portion is in said first position, and said passageway is in an open condition when said intermediate portion is in said second position.

2. The valve assembly of claim 1, wherein said intermediate portion comprises a valve control member for effecting said selective rotation between said first and second positions.

3. The valve assembly of claim 2, wherein said valve control member comprises a control knob graspable by an operator for effecting said selective rotation.

4. The valve assembly of claim 1, wherein said valve frame comprises a housing for said respective proximal, intermediate, and distal portions.

5. The valve assembly of claim 4, wherein said proximal portion and said distal portion are fixedly retained in said housing.

6. The valve assembly of claim 1, wherein each of said first and second valve members comprises a generally cylindrical sleeve.

7. The valve assembly of claim 6, wherein each of said sleeves comprises an elastomer.

8. The valve assembly of claim 1, wherein an inner surface of at least one of said sleeves includes a lubricious coating.

9. The valve assembly of claim 1, wherein said proximal portion comprises a large diameter portion and a small diameter portion, said small diameter portion extending distally from said large diameter portion and including a surface for receiving said first valve member proximal end; and
wherein said distal portion comprises a large diameter portion and a small diameter portion, said small diameter portion extending proximally from said large diameter portion and including a surface for receiving said first valve member distal end.

10. The valve assembly of claim 9, wherein said intermediate portion comprises a large diameter middle portion, and respective small diameter proximal and distal portions extending axially from said middle portion; said small diameter proximal portion configured and aligned for receiving said first valve member distal end, and said small diameter distal portion configured and aligned for receiving said second member proximal end.

11. The valve assembly of claim 1, wherein said intermediate portion comprises a large diameter middle portion, and respective small diameter proximal and distal portions extending axially from said large diameter middle portion; said small diameter proximal portion configured and aligned for receiving said first valve member distal end, and said small diameter distal portion configured and aligned for receiving said second member proximal end.

12. The valve assembly of claim 11, wherein said middle portion comprises a valve control member for effecting said selective rotation between said first and second positions.

13. A dual valve assembly for controlling a flow of fluid, comprising:
a housing comprising a proximal hub member, an intermediate hub member, and a distal hub member, said proximal hub member, intermediate hub member, and distal hub member configured and aligned in an interior space of said housing for passage of an interventional device, said proximal hub member and said distal hub member having a substantially fixed orientation in said housing interior space, said intermediate hub member rotatable between a first position and a second position in said housing interior space;
a first valve sleeve having a proximal end, a distal end, and a passageway therebetween for passage of said interventional device, said proximal end secured to said proximal hub member, and said distal end secured to said intermediate hub member, said first valve sleeve arranged relative to said proximal hub member and intermediate hub member such that said passageway is in an open condition for passage of said interventional device when said intermediate hub member is in said first position, and said passageway sealingly constricts to a closed condition when said intermediate hub member is in said second position; and
a second valve sleeve having a proximal end, a distal end, and a passageway therebetween for passage of said interventional device, said proximal end secured to said intermediate hub member, and said distal end secured to said valve frame distal hub member, said second valve sleeve arranged relative to said valve frame distal hub member and said intermediate hub member such that said passageway sealingly constricts to a closed condition when said intermediate hub member is in said first position, and said passageway is in an open condition for passage of said interventional device when said intermediate hub member is in said second position.

14. The dual valve assembly of claim 13, wherein said intermediate hub member comprises a valve control member for effecting said selective rotation between said first and second positions.

15. The dual valve assembly of claim 13, wherein said proximal hub member comprises a large diameter portion and a small diameter portion, said small diameter portion extending distally from said large diameter portion and including a surface for receiving said first valve sleeve proximal end; and
wherein said distal hub member comprises a large diameter portion and a small diameter portion, said small diameter portion extending proximally from said large diameter portion and including a surface for receiving said first valve sleeve distal end.

16. The dual valve assembly of claim 15, wherein said intermediate hub member comprises a large diameter middle portion, and respective small diameter proximal and distal portions extending axially from said middle portion;
said small diameter proximal portion configured and aligned for receiving said first valve sleeve distal end, and said small diameter distal portion configured and aligned for receiving said second sleeve proximal end.

17. The dual valve assembly of claim 13, wherein said intermediate hub member comprises a large diameter middle portion, and respective small diameter proximal and distal portions extending axially from said middle portion;
said small diameter proximal portion configured and aligned for receiving said first valve member distal end, and said small diameter distal portion configured and aligned for receiving said second member proximal end.

18. The dual valve assembly of claim 17, wherein said middle portion comprises a valve control member for effecting said selective rotation between said first and second positions.

19. The dual valve assembly of claim 18, wherein the housing comprises a slot extending therealong, and wherein said valve control member rides along said slot during movement of said sleeves between respective open and closed conditions.

20. The dual valve assembly of claim 19, wherein the housing has a generally cylindrical configuration, and wherein said slot extends along about one-half of the circumference of the housing.
